(19) United States
(12) Patent Application Publication
(10) Pub. No.: US 2019/0223826 A1
(43) Pub. Date: Jul. 25, 2019

(54) X-RAY CT MEASURING APPARATUS AND CALIBRATION METHOD THEREOF

(71) Applicant: MITUTOYO CORPORATION, Kanagawa (JP)

(72) Inventors: Hidemitsu ASANO, Kanagawa (JP); Masato KON, Kanagawa (JP)

(73) Assignee: MITUTOYO CORPORATION, Kanagawa (JP)

(21) Appl. No.: 16/250,201

(22) Filed: Jan. 17, 2019

(30) Foreign Application Priority Data
Jan. 19, 2018 (JP) 2018-007744

(51) Int. Cl. A61B 6/00; A61B 6/03; G01T 7/00; G01N 23/046

(52) U.S. Cl. CPC A61B 6/584; A61B 6/03; G01N 2223/419; G01T 7/005; G01N 23/046; A61B 6/40

(57) ABSTRACT

An X-ray CT measuring apparatus configured to emit an X-ray from an X-ray source while rotating a subject arranged on a rotary table, and obtain a tomographic image of the subject by reconstructing projection images, includes an X-ray fluctuation calibration jig arranged in an X-ray field of view, a detection unit configured to detect fluctuations in an X-ray focal position by using an X-ray projected image of the X-ray fluctuation calibration jig, and a correction unit configured to correct an X-ray projection image of the subject by using the detected fluctuations in the X-ray focal position.

### CROSS-REFERENCE TO RELATED APPLICATION

The disclosure of Japanese Patent Application No. 2018-007744 filed on Jan. 19, 2018 including specifications, drawings and claims are incorporated herein by reference in its entirety.

### TECHNICAL FIELD

The present invention relates to an X-ray CT measuring apparatus and a calibration method thereof. In particular, the present invention relates to an X-ray CT measuring apparatus capable of calibrating fluctuations in an X-ray focal position to obtain a high-quality tomographic image, and a calibration method thereof.

### BACKGROUND ART

An X-ray CT measuring apparatus configured to obtain a tomographic image of a subject (measurement object) in a nondestructive manner has been known (see Japanese Patent Application Laid-Open Nos. 2002-71345 and 2004-12407). The X-ray CT measuring apparatus performs X-ray irradiation while rotating the subject which is arranged at the center of a rotary table.

FIG. 1 shows a configuration of a typical X-ray CT apparatus used for measurement. An X-ray tube 12, an X-ray detector 14, a rotary table 16, and an XYZ moving mechanism unit 18 are accommodated in an enclosure 10 which shields X-rays. The X-ray tube 12 emits X-rays 13. The X-ray detector 14 detects the X-rays 13. A subject 8 is placed on the rotary table 16, and the rotary table 16 rotates the subject 8 for CT imaging. The XYZ moving mechanism unit 18 is intended to adjust the position and magnification of the subject 8 captured by the X-ray detector 14. The X-ray CT apparatus further includes a controller 20 which controls the devices, and a control personal computer (PC) 22 which gives instructions to the controller 20 on the basis of user operations.

Aside from the function of controlling the devices, the control PC 22 has a function of displaying a projection image of the subject 8 captured by the X-ray detector 14 and a function of reconstructing a tomographic image from a plurality of projection images of the subject 8.

When the X-rays 13 pass through an object, there occurs a considerable amount of scattered X-rays reflected in directions different from the irradiation direction. Such scattered X-rays are known to appear as noise in an X-ray CT imaging result. To suppress the scattered X-rays, an X-ray collimator 24 is arranged near the X-ray tube 12. To limit the irradiation range of the X-rays in a vertical direction, the X-ray collimator 24 includes parts, or an upper movable part 24A and a lower movable part 24B, that are made of a radiopaque material (such as tungsten). The parts 24A and 24B are each configured to be vertically movable. The positions of the upper and lower movable parts 24A and 24B of the X-ray collimator 24 are controlled by the control PC 22 according to the imaging range of the subject 8.

As shown in FIG. 2, the X-rays 13 emitted from an X-ray source including the X-ray tube 12 are transmitted through the subject 8 on the rotary table 16 and reach the X-ray detector 14. The X-ray detector 14 obtains transmission images (projection images) of the subject 8 in all directions while rotating the subject 8. The transmission images are reconstructed to generate a tomographic image of the subject 8.

The position of the subject 8 can be moved by controlling X-, Y-, and Z-axes of the XYZ moving mechanism unit 18 and a θ-axis of the rotary table 16. The imaging range (position and magnification) and the imaging angle of the subject 8 can be thereby adjusted.

The X-rays 13 are generated by collision of an electron beam with a target in the X-ray tube 12. Specifically, as shown in FIG. 3, a voltage (tube voltage) and a current (tube current) are supplied to the X-ray tube 12 to heat a filament 12A, whereby an electron beam 12B is generated. The electron beam 12B collides with a target 12C, and part of the energy is emitted from an X-ray irradiation window 12E as the X-rays 13. The colliding position of the electron beam with the target 12C will be referred to as an X-ray focus 12D. The stability and size of the X-ray focus 12D are closely related to the precision of the projection images of the subject 8 and the final tomographic image. The size of the X-ray focus 12D depends on the magnitudes of the tube voltage and the tube current. To obtain high-resolution image quality, the tube voltage and the tube current need to be adjusted to not increase the focus size. The focal position depends on the temperatures of the filament 12A and the target 12C, as well as the tube voltage and the tube current. The stability of the X-ray focal position is usually ensured by an X-ray tube warm-up and the like prior to the use of the X-rays. However, minute fluctuations in the focal position of the X-rays and movements due to temperature variations are unable to be completely removed. Fluctuations and movements of the focal position change the position and magnification of a projected image obtained by X-ray irradiation.

Japanese Patent No. 5408873 (paragraph 0084, and FIG. 8) describes that a reference 50 is arranged on the rotary table 60 for the sake of calibration. Japanese Patent Application Laid-Open No. 2002-55062 (claim 1, paragraph 0012, and FIGS. 1 and 2) describes that a sample table 10 equipped with a calibration jig is attached to a rotary table 3a and a sample 11 is set at the center.

### SUMMARY OF THE INVENTION

### Technical Problem

The techniques described in Japanese Patent No. 5408873 and Japanese Patent Application Laid-Open No. 2002-55062 are both intended to calibrate a deviation of the rotation center position of the rotary table, and have difficulty in detecting or calibrating fluctuations in the X-ray focal position. In particular, the technique described in Japanese Patent No. 5408873 has problems such as the need to replace the reference with the subject.

The present invention has been made in order to solve the above-described problems in the conventional technique, and an object thereof is to enable easy calibration of fluctuations in the X-ray focal position without replacement of a subject with a calibration jig.

### Solution to Problem

The present invention has solved the foregoing problems by the provision of an X-ray CT measuring apparatus configured to emit an X-ray from an X-ray source arranged on one side of a rotary table while rotating a subject arranged on the rotary table, and obtain a tomographic image of the subject by reconstructing projection images obtained by an X-ray detector arranged on an opposite side of the rotary table, the X-ray CT measuring apparatus including: an X-ray fluctuation calibration jig arranged in an X-ray field of view; a detection unit configured to detect fluctuations in an X-ray focal position by using an X-ray projected image of the X-ray fluctuation calibration jig; and a correction unit configured to correct an X-ray projection image of the subject by using the detected fluctuations in the X-ray focal position.

Herein, the X-ray fluctuation calibration jig may be an X-ray shielding frame arranged to surround the X-ray projected image of the subject on the X-ray projection image.

The X-ray shielding frame may be rectangular in shape and arranged so that an entire window opened by the X-ray shielding frame is projected on the X-ray detector.

The window may have a width and height of calibrated lengths. The X-ray shielding frame may be made of a material having a low coefficient of thermal expansion.

The X-ray fluctuation calibration jig may be adjustable in position.

The X-ray fluctuation calibration jig may be an X-ray passing port formed in an X-ray collimator provided for limiting irradiation range of the X-rays.

The X-ray passing port may be a combination of a horizontally long slit and a vertically long slit.

The present invention also provides a calibration method of an X-ray CT measuring apparatus configured to emit an X-ray from an X-ray source arranged on one side of a rotary table while rotating a subject arranged on the rotary table, and obtain a tomographic image of the subject by reconstructing projection images obtained by an X-ray detector arranged on an opposite side of the rotary table, the calibration method including: arranging an X-ray fluctuation calibration jig in an X-ray field of view of the X-ray CT measuring apparatus; detecting fluctuations in an X-ray focal position by using an X-ray projected image of the X-ray fluctuation calibration jig; and correcting an X-ray projection image of the subject by using the detected fluctuations in the X-ray focal position.

The fluctuations in the X-ray focal position may be detected by comparing positions and sizes of a window formed by the X-ray fluctuation calibration jig in the respective projection images with reference to a position and size of the window at a point in time.

A vertex of a frame window opened by an X-ray shielding frame of the X-ray fluctuation calibration jig and determined from a first projection image may be assumed as a reference vertex, and second and subsequent projection images may be corrected by using the reference vertex.

The projection images may be corrected by using an affine transformation.

An actual projection magnification may be calculated by determining a physical projected length of the frame window from two adjoining reference vertexes. A projection magnification may be corrected by determining an offset of an X-ray source position, using the actual projection magnification.

### Advantageous Effects of Invention

Fluctuations in the X-ray focal position in each projection image can be calibrated by using the dedicated X-ray fluctuation calibration jig permanently provided on the X-ray CT measuring apparatus. A tomographic image of higher quality can thus be obtained by correcting the effect of fluctuations of the X-ray focus on the projection images.

### BRIEF DESCRIPTION OF THE DRAWINGS

The preferred embodiments will be described with reference to the drawings, wherein like elements have been denoted throughout the figures with like reference numerals, and wherein;

FIG. 1 is a cross-sectional view showing an overall configuration of a typical X-ray CT apparatus used for measurement;

FIG. 2 is a perspective view showing an arrangement of essential parts thereof;

FIG. 3 is a cross-sectional view showing a principle of generation of X-rays thereof;

FIG. 4 is a cross-sectional view showing an overall configuration of a first embodiment of an X-ray CT measuring apparatus according to the present invention;

FIG. 5 is a perspective view showing an arrangement of essential parts thereof;

FIG. 6 is a diagram showing an example of a projected image of an X-ray fluctuation calibration jig projected on an X-ray detector thereof;

FIG. 7 is a flowchart showing a calibration procedure according to the first embodiment;

FIG. 8 is a diagram showing a method for processing a projection image;

FIG. 9 is a diagram showing a state in which the projection image is corrected;

FIG. 10 is a diagram showing a positional relationship for magnification correction;

FIG. 11 is a diagram showing X-ray passing ports serving as an X-ray fluctuation calibration jig used in a second embodiment of the present invention; and

FIG. 12 is a diagram showing a projected image of the X-ray passing ports according to the second embodiment.

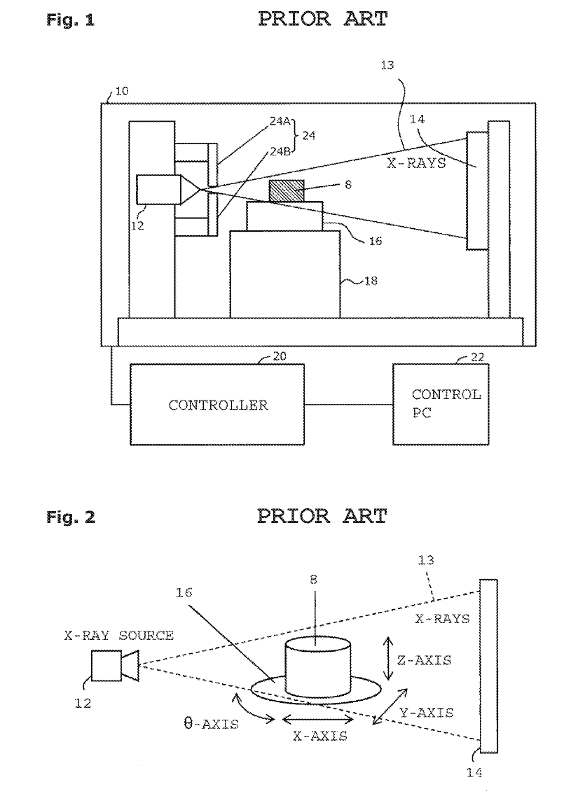

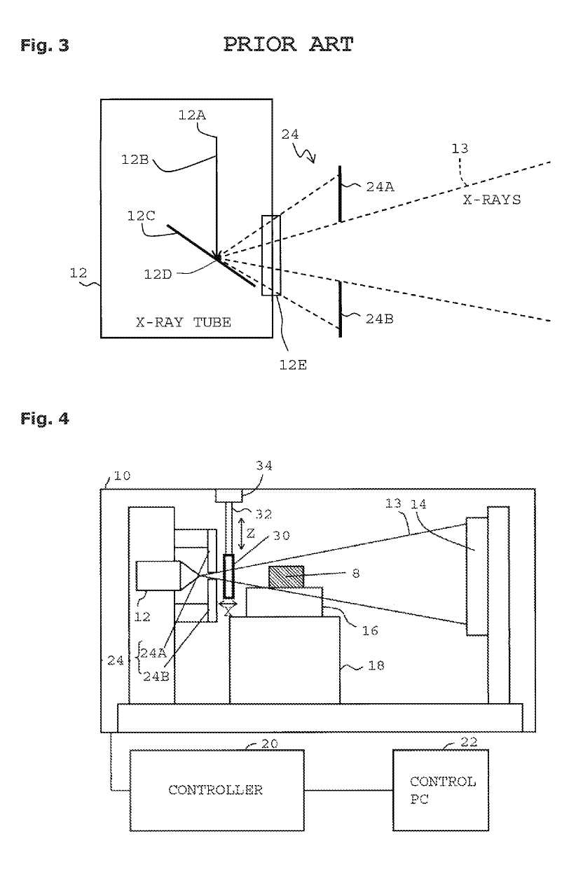

### DESCRIPTION OF EMBODIMENTS

With reference to the drawings, an embodiment of the present invention will be described below in more detail. Note that the present invention is not to be limited by the contents described in the following embodiments and examples. Additionally, the components in the embodiments and examples described below include those one skilled in the art can readily conceive or being substantially the same, i.e., the so-called equivalents. Furthermore, the components disclosed in the embodiments and examples described below may be combined as appropriate or may also be selected as appropriate for use.

The present invention achieves generation of a more precise tomographic image by permanently providing a dedicated X-ray fluctuation calibration jig on an X-ray CT measuring apparatus and correcting the effect of fluctuations of an X-ray focal position appearing on an image each time an X-ray projection image is obtained.

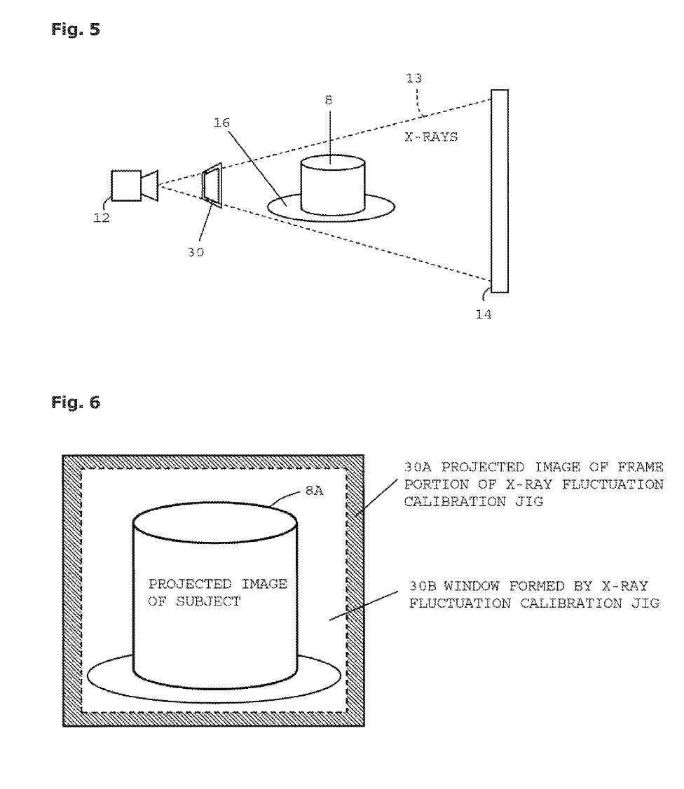

As shown in FIG. 4 (cross-sectional view showing an overall configuration) and FIG. 5 (perspective view showing an arrangement of essential parts), a first embodiment of the present invention includes an X-ray fluctuation calibration jig 30 of frame shape between an X-ray collimator 24 and a rotary table 16. The X-ray fluctuation calibration 30 is arranged to surround a projected image 8A of a subject 8 on a projection image as illustrated in FIG. 6 and shields X-rays 13.

The X-ray fluctuation calibration jig 30 includes a rectangular frame that shields the X-rays 13. As shown in FIG. 6, the X-ray fluctuation calibration jig 30 is arranged so that the entire window opened by the frame is projected on an X-ray detector 14. In the diagram, 30A represents the projected image of the frame portion of the X-ray fluctuation calibration jig 30, and 30B the window formed by the frame of the X-ray fluctuation calibration jig 30.

The window has a width and height of calibrated lengths, and the frame is made of a material having a low coefficient of thermal expansion. With such a structure, the window is less likely to change in width or height even the temperature of the frame is increased by X-ray irradiation.

The X-ray fluctuation calibration jig 30 is hung from the ceiling of an enclosure 10 by a stay 32. The stay 32 is provided with a position adjustment mechanism 34, and the position of the X-ray fluctuation calibration jig 30 can be adjusted vertically (Z-axis direction in FIG. 2), horizontally (Y-axis direction), and in a front-to-back direction (X-axis direction) according to need. The position adjustment mechanism 34 may be omitted so that the stay 32 is fixed to the enclosure 10.

In FIG. 4, the X-ray fluctuation calibration jig 30 is installed near an X-ray tube 12. However, the X-ray fluctuation calibration jig 30 can be installed at any position as long as the entire window can be projected on the X-ray detector 14. The frame is not limited to the rectangular shape, either, as long as calibrated lengths of the X-ray fluctuation calibration jig 30 can be identified on the projection image.

FIG. 6 shows the projected image of the X-ray fluctuation calibration jig 30 projected on the X-ray detector 14. The projected image 30A of the frame portion of the X-ray fluctuation calibration jig 30 is projected on the X-ray detector 14 by X-ray irradiation. Since the frame portion of the X-ray fluctuation calibration jig 30 does not transmit X-rays, the projected image 30A of the frame portion on the X-ray detector 14 has the same value as when the X-rays are off (gray level). The position and size of the window (projected image 30B) formed by the X-ray fluctuation calibration jig 30 can thus be detected.

For example, if the X-ray focal position fluctuates upward in the Z-axis direction in parallel with the detector plane, the projected image 30B of the window is displayed as shifted down in the Z-axis direction. If the X-ray focal position fluctuates toward the X-ray source in the X-axis direction, the projected image 30B of the window is displayed as enlarged.

Fluctuations in the X-ray focal position can thus be detected by comparing the positions and sizes of the window in respective projection images with reference to the position and size of the window at a point in time. Calibration can be performed by using the amounts of change.

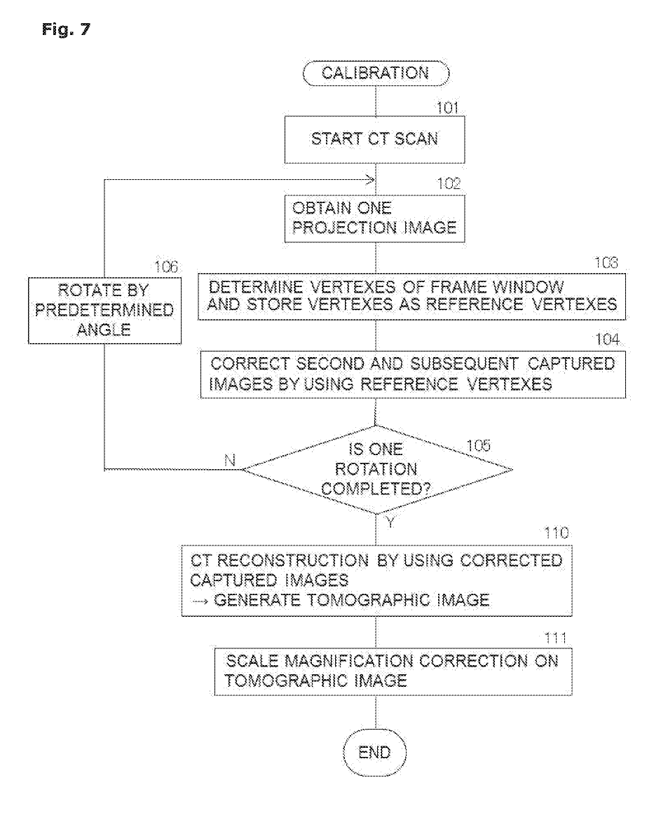

An embodiment of a specific calibration procedure will be described below with reference to FIG. 7.

Initially, in step 101, a CT scan is started.

Next, in step 102, one projection image is obtained.

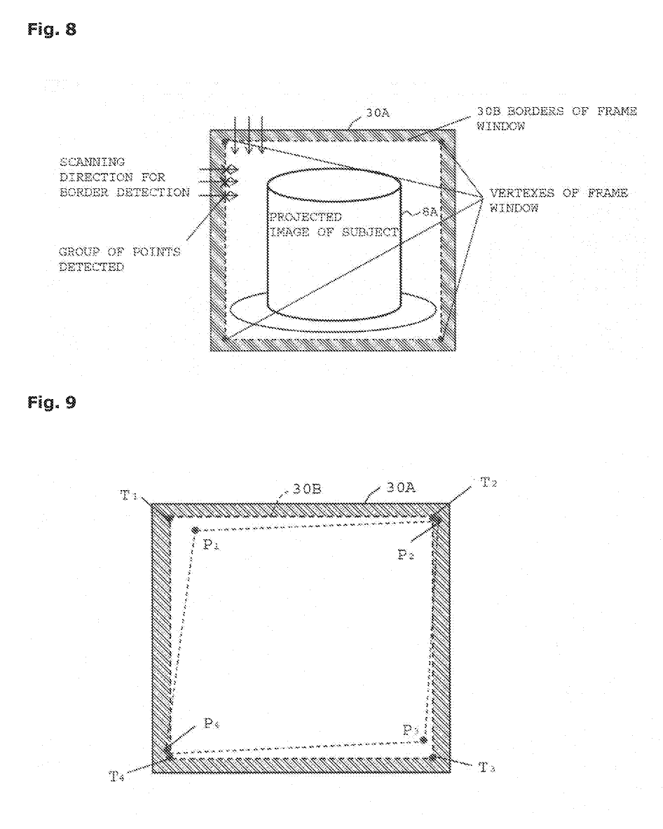

Next, in step 103, the vertexes of the frame window are determined by resolution analysis of the projection image. Specifically, to determine the borders of the frame window in the projection image, the projection image is initially scanned from ends to inward as illustrated in FIG. 8. A group of points where luminance with X-rays off changes to luminance with X-rays on (or to or above a threshold) are thereby detected. As a specific algorithm, an edge detection function of image processing can be used.

Using the group of points detected, the positions of the four sides of the frame window on the projection image are determined by fitting (geometric element fitting). The intersections of the sides (vertexes of the frame window) are then calculated.

The vertexes of the frame window determined from the first projection image are stored as reference vertexes.

Next, in step 104, second and subsequent projection images are corrected as illustrated in FIG. 9 by using the reference vertexes.

For example, the correction uses an affine transformation. Coordinates T_{N}(x′_{N}, y′_{N}) of each reference vertex and coordinates P_{N}(x_{N}, y_{N}) of each vertex of the frame window, calculated from a projection image, can be expressed by using an affine transformation as follows:

$$[ \begin{matrix}{x}_{N}^{′} \\ {y}_{N}^{′} \\ 1\end{matrix} ] = [ \begin{matrix}a & b & s \\ c & d & t \\ 0 & 0 & 1\end{matrix} ] [ \begin{matrix}{x}_{N} \\ {y}_{N} \\ 1\end{matrix} ] \qquad ( 1 )$$

Here, the parameters a, b, c, d, s, and t of the transformation matrix are determined from the actual coordinates of the reference vertexes and the vertexes of the frame window. A calculation formula for correcting an arbitrary point P of a projection image to a corrected position P′ is then derived.

$$\begin{matrix}{P}^{′} = MP & ( 2 ) \\ M = [ \begin{matrix}a & b & s \\ c & d & t \\ 0 & 0 & 1\end{matrix} ] & ( 3 )\end{matrix}$$

An entire projection image obtained can be corrected by applying the foregoing calculation to each pixel of the projection image. This correction processing may include interpolation processing if needed.

Next, in step 105, whether the rotary table 16 has completed one rotation is determined. Until one rotation is completed, the rotary table 16 is rotated in units of a predetermine angle in step 106, and the processing of steps 102 to 104 is performed at each rotation angle of the CT scan.

If the rotary table 16 is determined to have completed one rotation in step 105, the processing proceeds to step 110. In step 110, CT reconstruction is performed by using the corrected projection images, whereby a tomographic image is generated.

In step 111, a scale magnification correction is made on the tomographic image.

Specifically, a physical projected length of the frame window is initially determined from two adjoining reference vertexes, and an actual projection magnification Mag′ is calculated. For example, suppose that the width of the frame window is 2.0 mm, the distance between adjoining reference vertexes of the frame window on a projection image is 1001 pixels, and the pixel pitch of the X-ray detector is 0.2 mm/pixel. In such a case, the physical length on the projection image is 200.2 mm, and the projection magnification is 100.1 times.

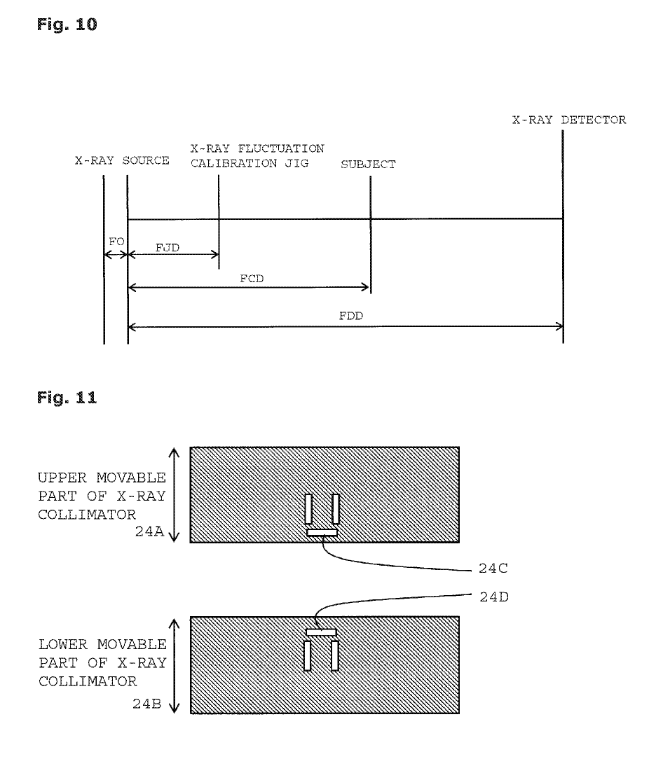

As illustrated in FIG. 10, a theoretical distance between the X-ray source and the X-ray detector will be denoted by FDD (Focus to Detector Distance), and a theoretical distance between the X-ray source and the X-ray fluctuation calibration jig by FJD (Focus to Jig Distance). A theoretical projection magnification Mag of the jig is then given as follows:

$$Mag = \frac{FDD}{FJD} \qquad ( 4 )$$

An offset between a theoretical X-ray source position and an actual X-ray source position will be denoted by FO (Focus Offset). The actual projection magnification Mag′ is then expressed as follows:

$${Mag}^{′} = \frac{FDD + FO}{FJD + FO} \qquad ( 5 )$$

From the foregoing equation, the offset FO of the X-ray source position can be determined by using the initially-calculated Mag′.

For example, if FDD=1000 mm and FJD=10 mm, the theoretical projection magnification Mag=100. If the actual projection magnification Mag′=100.1 as in the foregoing example, FO=−1/99.1≈−0.01 mm from the foregoing calculation formula.

The scale of the tomographic image per pixel is given by S/M [mm], where S [mm/pixel] is the pixel pitch of the X-ray detector 14 and M is the projection magnification of the subject 8. Magnification correction can be made by using the following projection magnification M′ in consideration of the offset of the X-ray source position:

$${M}^{′} = \frac{FDD + FO}{FCD + FO} \qquad ( 6 )$$

In the present embodiment, the X-ray fluctuation calibration jig 30 can be arranged at a desirable position suited for calibration.

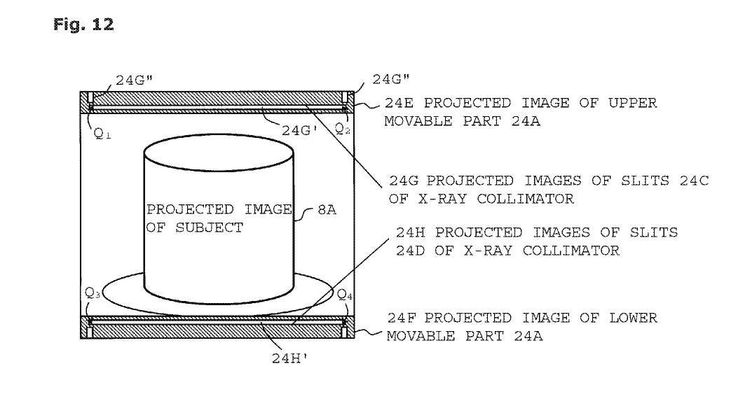

Next, a second embodiment of the present invention will be described. In this embodiment, the X-ray fluctuation calibration jig 30 of frame shape is substituted by X-ray passing slits 24C and 24D which are formed in the upper movable part 24A and the lower movable part 24B of the X-ray collimator 24 as shown in FIG. 11, respectively. The X-ray passing slits 24C and 24D serve as X-ray passing ports through which the X-rays 13 can pass. In other words, the slender X-ray passing slits 24C and 24D are formed in the upper and lower movable parts 24A and 24B of the X-ray collimator 24 so that the X-rays can pass through. As shown in FIG. 12, the slits 24C and 24D are configured so that their projected images 24G and 24H can be projected on the X-ray detector 14. In the diagram, 24E and 24F represent projected images of the upper movable part 24A and the lower movable part 24B of the X-ray collimator 24, respectively.

In this example, a horizontally long slit 24G′ and vertically long slits 24G″ are detected from the projected image 24E of the upper movable part 24A, and the positions of the respective slits are determined. End points Q₁ and Q₂ of the horizontally long slit 24G′ are calculated by intersection calculation. Similar calculations are performed on the projected image 24F of the lower movable part 24B to determine end points Q₃ and Q₄ of a horizontally long slit 24H′. Correction processing similar to the case with the X-ray fluctuation calibration jig 30 can be performed by using the resulting four end points Q₁ to Q₄.

The sizes and positions of the slits 24C and 24D are determined in consideration of the projection magnification and the projection size of the subject 8 during the CT scan. Attention needs to be paid since the slits can be too large in size to fall within the range of a projection image or too small to be displayed on a projection image, depending on the projection magnification. The slits are desirably located at the X-ray irradiation port side of the x-ray collimator 24 (a lower side of the upper movable part 24A and an upper side of the lower movable part 24B). Otherwise, the projection image includes the projected images 24E and 24F of the X-ray collimator 24 more than necessary, and the display range of the projection image 8A of the subject 8 is narrowed.

The slits may have any shape (such as a rectangular shape and a pinhole shape) as long as slit portions having calibrated lengths can be detected from the projection image. For more accurate position calculation, a plurality of geometric shapes (rectangles, perfect circles) can be detected for geometrical calculation such as intersection calculation.

The second embodiment has a simple configuration since, unlike the first embodiment, the separate X-ray fluctuation calibration jig 30 does not need to be provided.

It should be apparent to those skilled in the art that the above-described embodiments are merely illustrative which represent the application of the principles of the present invention. Numerous and varied other arrangements can be readily devised by those skilled in the art without departing from the spirit and the scope of the invention.

1. An X-ray CT measuring apparatus configured to emit an X-ray from an X-ray source arranged on one side of a rotary table while rotating a subject arranged on the rotary table, and obtain a tomographic image of the subject by reconstructing projection images obtained by an X-ray detector arranged on an opposite side of the rotary table, the X-ray CT measuring apparatus comprising:
an X-ray fluctuation calibration jig arranged in an X-ray field of view;
a detection unit configured to detect fluctuations in an X-ray focal position by using an X-ray projected image of the X-ray fluctuation calibration jig; and
a correction unit configured to correct an X-ray projection image of the subject by using the detected fluctuations in the X-ray focal position.

2. The X-ray CT measuring apparatus according to claim 1, wherein the X-ray fluctuation calibration jig is an X-ray shielding frame arranged to surround the X-ray projected image of the subject on the X-ray projection image.

3. The X-ray CT measuring apparatus according to claim 2, wherein the X-ray shielding frame is rectangular in shape and arranged so that an entire window opened by the X-ray shielding frame is projected on the X-ray detector.

4. The X-ray CT measuring apparatus according to claim 3, wherein the window has a width and height of calibrated lengths, and the X-ray shielding frame is made of material having a low coefficient of thermal expansion.

5. The X-ray CT measuring apparatus according to claim 1, wherein the X-ray fluctuation calibration jig is adjustable in position.

6. The X-ray CT measuring apparatus according to claim 1, wherein the X-ray fluctuation calibration jig is an X-ray passing port formed in an X-ray collimator provided for limiting irradiation range of the X-rays.

7. The X-ray CT measuring apparatus according to claim 6, wherein the X-ray passing port is a combination of a horizontally long slit and a vertically long slit.

8. A calibration method of an X-ray CT measuring apparatus configured to emit an X-ray from an X-ray source arranged on one side of a rotary table while rotating a subject arranged on the rotary table, and obtain a tomographic image of the subject by reconstructing projection images obtained by an X-ray detector arranged on an opposite side of the rotary table, the calibration method comprising:
arranging an X-ray fluctuation calibration jig in an X-ray field of view of the X-ray CT measuring apparatus;
detecting fluctuations in an X-ray focal position by using an X-ray projected image of the X-ray fluctuation calibration jig; and
correcting an X-ray projection image of the subject by using the detected fluctuations in the X-ray focal position.

9. The calibration method of an X-ray CT measuring apparatus according to claim 8, wherein the fluctuations in the X-ray focal position are detected by comparing positions and sizes of a window formed by the X-ray fluctuation calibration jig in the respective projection images with reference to a position and size of the window at a point in time.

10. The calibration method of an X-ray CT measuring apparatus according to claim 8, wherein a vertex of a frame window opened by an X-ray shielding frame of the X-ray fluctuation calibration jig and determined from a first projection image is assumed as a reference vertex, and second and subsequent projection images are corrected by using the reference vertex.

11. The calibration method of an X-ray CT measuring apparatus according to claim 10, wherein the projection images are corrected by using an affine transformation.

12. The calibration method of an X-ray CT measuring apparatus according to claim 10, wherein an actual projection magnification is calculated by determining a physical projected length of the frame window from two adjoining reference vertexes, and a projection magnification is corrected by determining an offset of an X-ray source position, using the actual projection magnification.
